(19) United States
(12) Patent Application Publication
(10) Pub. No.: US 2018/0374566 A1
(43) Pub. Date: Dec. 27, 2018

(54) SYSTEM FOR MONITORING LIQUID SUBSTANCE ADMINISTERED BY IV INFUSION SET

(71) Applicants: CHIH-WEI CHEN, New Taipei City (TW); HUI-YU CHEN, Taoyuan City (TW)

(72) Inventors: CHIH-WEI CHEN, New Taipei City (TW); HUI-YU CHEN, Taoyuan City (TW)

(21) Appl. No.: 16/016,523

(22) Filed: Jun. 22, 2018

(30) Foreign Application Priority Data
Jun. 27, 2017 (TW) 106209323

(51) Int. Cl. G16H 20/17; G06K 7/14; G06K 7/10; A61M 5/172

(52) U.S. Cl. CPC G16H 20/17; G06K 7/1413; G06K 7/10881; A61M 5/172; A61M 2205/3334; A61M 2205/6009; A61M 2205/3584; A61M 2205/3379; A61M 2205/6072

(57) ABSTRACT

A system for monitoring administered liquid substance includes a medicine administration system including an employee bar coded label, a patient bar coded label, a medicine bar coded label, and a medicine controller having a medicine controller bar coded label and a medicine administration device; and an administration monitoring system including a computer for medication, a barcode reader, and a handheld mobile device. The barcode reader scans the employee bar coded label, the patient bar coded label, the medicine bar coded label, and the medicine controller bar coded label and sends the read barcodes to the computer for medication; a volume of substance to be administered is set by the medicine controller based on the read barcodes; the medicine controller outputs the set volume of substance to the computer for medication; the computer for medication compares the set volume of substance with a volume of substance written on the EMR, and a comparison result is sent to the handheld mobile device for showing.

### BACKGROUND OF THE INVENTION

### 1. Field of the invention

The invention relates to intravenous (IV) infusion and more particularly to a system for monitoring liquid substance administered by an IV infusion set by comparing a set volume of substance with a volume of substance written on an electronic medical record (EMR) belonging to a target patient.

### 2. Description of Related Art

Conventionally, after going to a hospital and treated by a physician, a nurse may write a volume of substance on an IV bag based on an EMR. Next, the nurse may administer the liquid substance to a patient based on the volume of substance. However, it is often that the nurse may erroneously write the volume of substance on the IV bag due to carelessness. And in turn, the patient may be administered by too much or too little medicine. This error may be fatal in some cases.

There is a conventional IV infusion control system which is characterized below. A nurse uses a barcode reader to read printed barcodes on each of an employee's clothes, a patient's clothes, an IV bag and a medicine controller by scanning. An infusion rate is sent from a device to a flow control device by using Wi-Fi technology. The nurse activates the flow control device to begin the administration. While its operation is quick, it may be hacked. Further, its installation and operation costs are prohibitively high.

Thus, it is desirable of providing a system capable of monitoring liquid substance administered by an IV infusion set with improved accuracy.

### SUMMARY OF THE INVENTION

It is therefore one object of the invention to provide a system for monitoring administered liquid substance, comprising a medicine administration system comprising an employee bar coded label, a patient bar coded label, a medicine bar coded label, and a medicine controller including a medicine controller bar coded label and a medicine administration device wherein the employee bar coded label, the patient bar coded label, the medicine bar coded label, and the medicine controller bar coded label are arranged differently; and an administration monitoring system comprising a computer for medication, a barcode reader, and at least one handheld mobile device; wherein the computer for medication is stored with a plurality of EMRs and electrically connected to the barcode reader and the handheld mobile device respectively; and wherein the administration monitoring system is connected to the medicine controller via the Internet; wherein the barcode reader reads barcodes on each of the employee bar coded label, the patient bar coded label, the medicine bar coded label, and the medicine controller bar coded label and sends the read barcodes to the computer for medication; a volume of substance to be administered is set by the medicine controller based on the read barcodes sent from the computer for medication via the handheld mobile device; the medicine controller outputs the set volume of substance to the computer for medication; the computer for medication compares the set volume of substance with a volume of substance written on the EMR, and a result of the comparison is sent to the handheld mobile device for showing.

The invention has the following advantages: The computer for medicine compares a set volume of substance with a volume of substance written on an EMR belonging to a target patient. The system can monitor the administration in real time via the Internet. The invention is an exemplary embodiment of the Internet of Things (IoT). In contrast, the conventional art monitors the difference between a set volume of substance with a volume of substance written on an EMR belonging to a target patient is not done automatically (i.e., manually). Thus, the invention is not liable to error in the process of infusing liquid substance to a patient by using an IV infusion set in comparison with the conventional art.

### BRIEF DESCRIPTION OF THE DRAWINGS

The invention, as well as its many advantages, may be further understood by the following detailed description and drawings in which:

FIG. 1 schematically depicts the invention;

FIG. 2 is a block diagram according to a first preferred embodiment of the invention;

FIG. 3 is a block diagram according to a second preferred embodiment of the invention;

FIG. 4 is a block diagram according to a third preferred embodiment of the invention; and

FIG. 5 schematically depicts a wireless connection among the computer for medication, the barcode reader and the handheld mobile device.

### DETAILED DESCRIPTION OF THE INVENTION

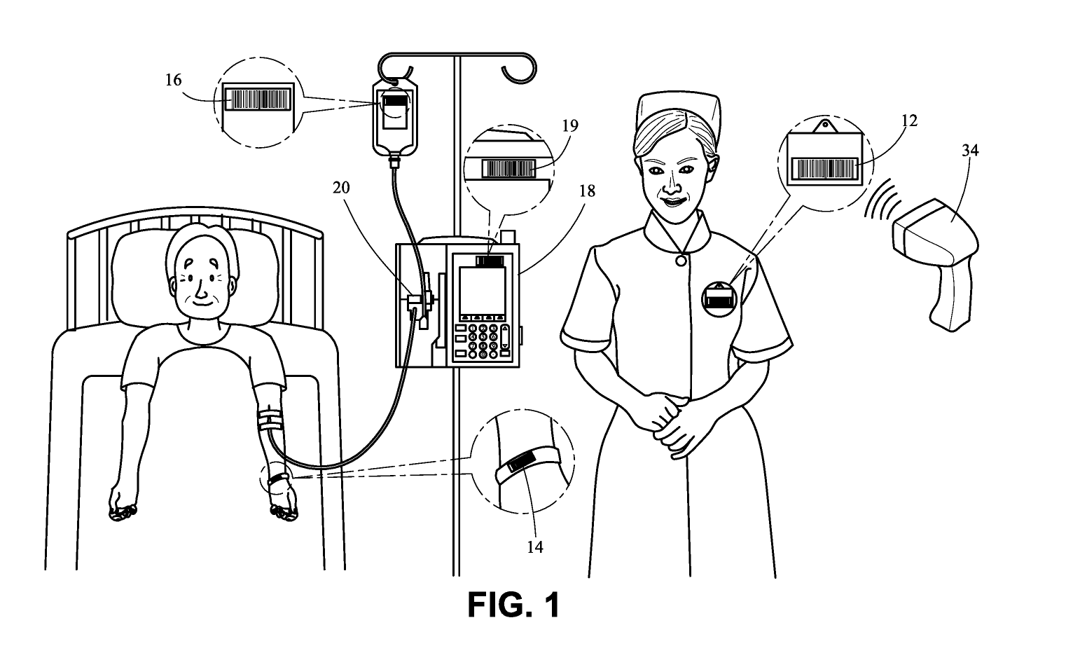

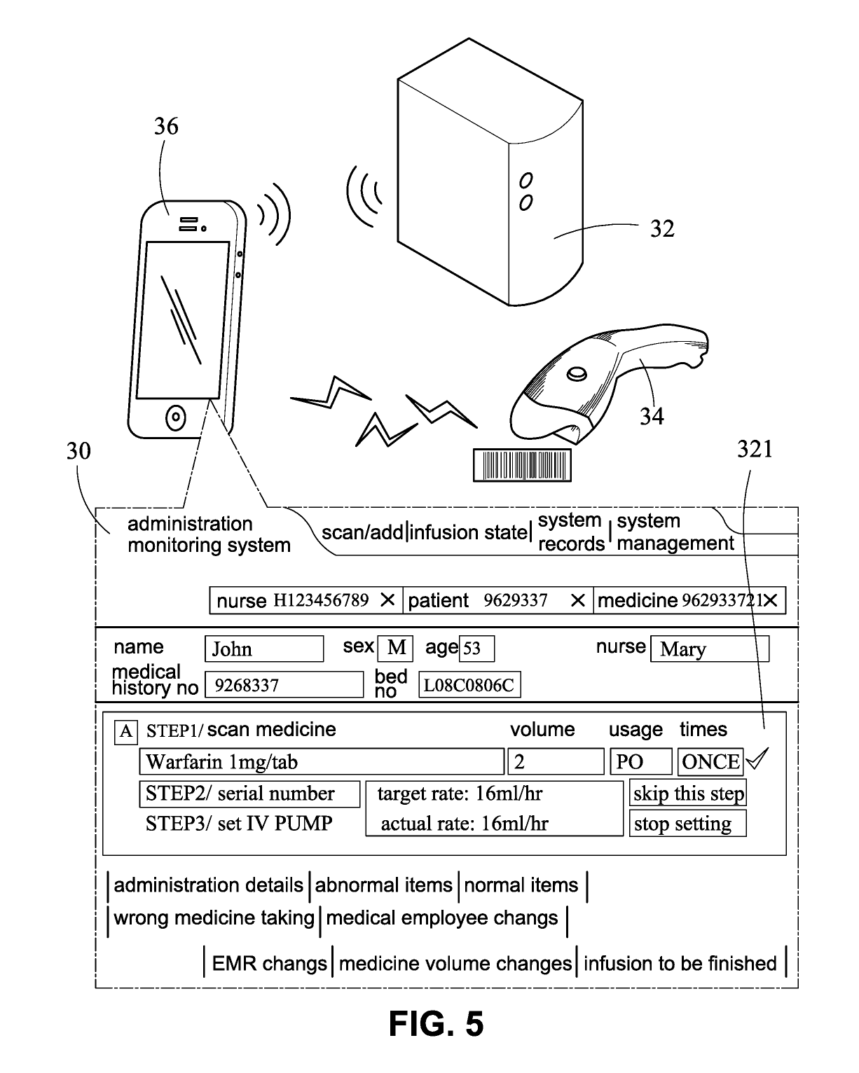

Referring to FIGS. 1 to 5, the invention comprises the following components as discussed in detail below.

A medicine administration system 10 comprises an employee bar coded label 12, a patient bar coded label 14, a medicine bar coded label 16 and a medicine controller 18 having a medicine controller bar coded label 19 provided thereon, and a medicine administration device 20. The employee bar coded label 12, the patient bar coded label 14, the medicine bar coded label 16 and the medicine controller bar coded label 19 are arranged differently.

An administration monitoring system 30 comprises a computer for medication 32, a barcode reader 34 and a handheld mobile device (e.g., a smart phone (as shown), a personal computer, a tablet computer, a personal digital assistant (PDA) reader, etc.) 36, The computer for medication 32 is stored with a plurality of EMRs 321 and electrically connected to the barcode reader 34 and the handheld mobile device 36 respectively. The administration monitoring system 30 is connected to the medicine controller 18 via the Internet. A predetermined volume of substance to be administered set by the medicine controller 18 is based on substance, volume, usage and times of administration written on the EMR 321. Details of the EMR 321 and printed barcodes read by the barcode reader 34 are shown on the handheld mobile device 36.

The handheld mobile device 36 can show IV infusion monitored by the administration monitoring system 30 in real time and an alert of “infusion to be finished” is sent to the handheld mobile device 36 for showing when the administration monitoring system 30 monitors same. Further, the handheld mobile device 36 can show system information, administration details, abnormal items, normal items, wrong medicine taking, medical employee changes, EMR changes, medicine volume changes, serial numbers, etc.

The barcode reader 34 is either a smart phone or a barcode scanner.

The medicine controller 18 is connected to the administration monitoring system 30 by wire or wirelessly.

The barcode reader 34, the handheld mobile device 36 and the computer for medication 32 are connected together by wire or wirelessly.

As shown in FIG. 1 specifically, the medicine controller 18 is a flow controller device for a drip chamber (not numbered).

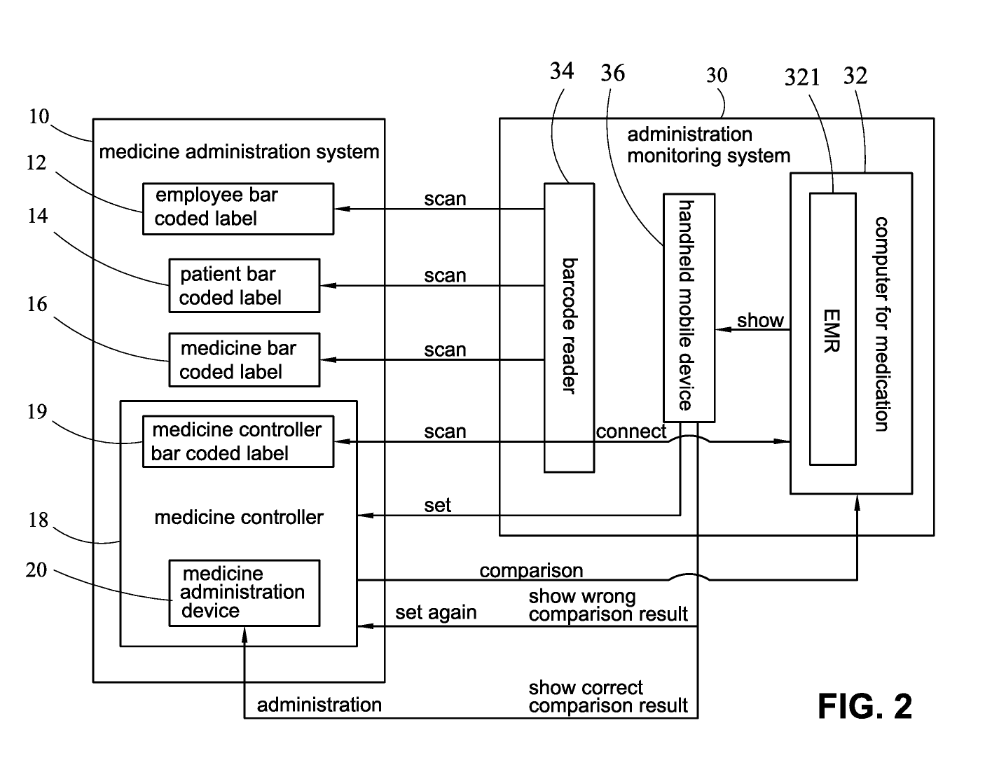

A first preferred embodiment of the invention is shown in FIG. 2 specifically. The characteristics of the first preferred embodiment are detailed below.

A medical employee may use the barcode reader 34 to read printed barcodes on each of the employee bar coded label 12, the patient bar coded label 14, the medicine bar coded label 16 and the medicine controller bar coded label 19 by scanning.

The read barcodes are sent from the barcode reader 34 to the computer for medication 32. The medical employee uses the handheld mobile device 36 to operate the medicine controller 18 which in turn sets a volume of substance to be administered based on the read barcodes sent from the computer for medication 32 via the handheld mobile device 36. The medical employee further uses the handheld mobile device 36 to operate the medicine controller 18 which in turn outputs the set volume of substance to the computer for medication 32. The computer for medication 32 compares the set volume of substance with a volume of substance written on the EMR 321 belonging to a target patient. The comparison result is sent to the handheld mobile device 36 for showing. The medical employee may use the handheld mobile device 36 to activate the medicine administration device 20 if the comparison result is correct. And in turn, the medicine administration device 20 administers medicine to the target patient. To the contrary, the medical employee may use the handheld mobile device 36 to operate the medicine controller 18 which in turn adjusts the set volume of substance to be equal to the volume of substance written on the EMR 321 if the comparison result is wrong. As an end, the medicine administration device 20 infuses liquid substance to the target patient.

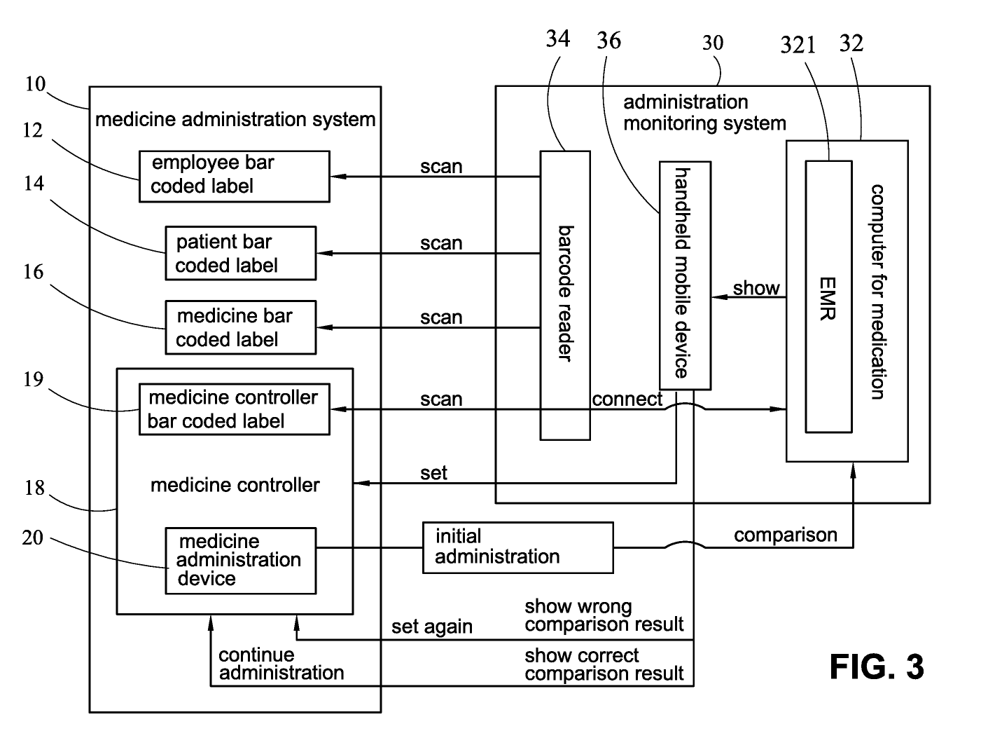

A second preferred embodiment of the invention is shown in FIG. 3 specifically. The characteristics of the second preferred embodiment are detailed below.

A medical employee may use the barcode reader 34 to read printed barcodes on each of the employee bar coded label 12, the patient bar coded label 14, the medicine bar coded label 16 and the medicine controller bar coded label 19 by scanning.

The read barcodes are sent from the barcode reader 34 to the computer for medication 32. The medical employee uses the handheld mobile device 36 to operate the medicine controller 18 which in turn sets a volume of substance to be administered based on the read barcodes sent from the computer for medication 32 via the handheld mobile device 36

The read barcodes are sent from the barcode reader 34 to the computer for medication 32. The medical employee uses the handheld mobile device 36 to operate the medicine controller 18 which in turn sets a volume of substance to be administered based on the read barcodes sent from the computer for medication 32 via the handheld mobile device 36

The medical employee further uses the handheld mobile device 36 to operate the medicine administration device 20 which in turn infuses liquid substance to a target patient and outputs the set volume of substance to the computer for medication 32. The computer for medication 32 compares the set volume of substance with a volume of substance written on the EMR 321 belonging to the target patient. The comparison result is sent to the handheld mobile device 36 for showing. The medicine administration device 20 continues to administer medicine if the comparison result is correct. To the contrary, the medical employee may use the handheld mobile device 36 to deactivate the medicine administration device 20. Next, the medical employee may use the handheld mobile device 36 to operate the medicine controller 18 which in turn adjusts the set volume of substance to be equal to the volume of substance written on the EMR 321 if the comparison result is wrong. As an end, the medicine administration device 20 activates again to infuse liquid substance to the target patient.

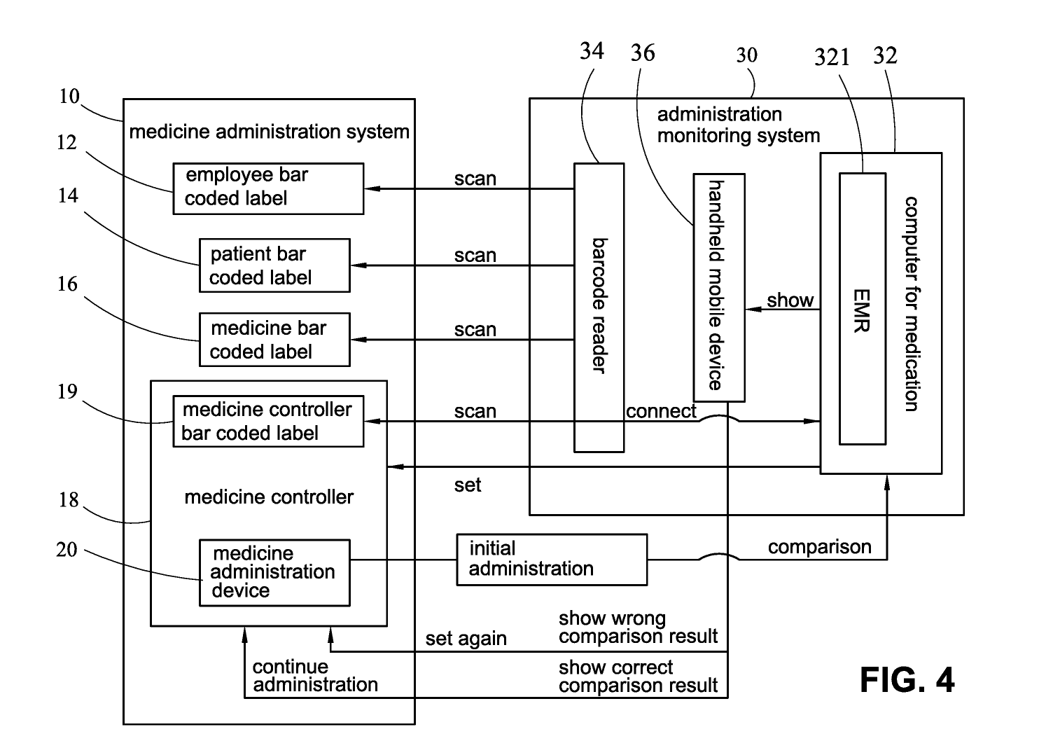

A third preferred embodiment of the invention is shown in FIG. 4 specifically. The characteristics of the third preferred embodiment are detailed below.

A medical employee may use the barcode reader 34 to read printed barcodes on each of the employee bar coded label 12, the patient bar coded label 14, the medicine bar coded label 16 and the medicine controller bar coded label 19 by scanning.

The read barcodes are sent from the barcode reader 34 to the computer for medication 32. The medical employee operates the computer for medication 32 which in turn activates the medicine controller 18 by using Wi-Fi technology. Then the medicine controller 18 sets a volume of substance to be administered based on the read barcodes sent from the computer for medication 32. The medical employee further uses the handheld mobile device 36 to operate the medicine administration device 20 which in turn infuses liquid substance to a target patient and outputs the set volume of substance to the computer for medication 32. The computer for medication 32 compares the set volume of substance with a volume of substance written on the EMR 321 belonging to the target patient. The comparison result is sent to the handheld mobile device 36 for showing. The medicine administration device 20 continues to administer medicine if the comparison result is correct. To the contrary, the medical employee may use the handheld mobile device 36 to deactivate the medicine administration device 20. Next, the medical employee may use the handheld mobile device 36 to operate the medicine controller 18 which in turn adjusts the set volume of substance to be equal to the volume of substance written on the EMR 321 if the comparison result is wrong. As an end, the medicine administration device 20 activates again to infuse liquid substance to the target patient.

It is envisaged by the invention that the system for monitoring liquid substance administered by an IV infusion set of the invention is characterized below. The computer for medicine compares a set volume of substance with a volume of substance written on an EMR belonging to a target patient. The system can monitor the administration in real time via the Internet. The invention is an exemplary embodiment of the Internet of Things (IoT). In contrast, the conventional art monitors the difference between a set volume of substance with a volume of substance written on an EMR belonging to a target patient is not done automatically (i.e., manually). Thus, the invention is not liable to error in the process of infusing liquid substance to a patient by using an IV infusion set in comparison with the conventional art.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A system for monitoring administered liquid substance, comprising:
a medicine administration system comprising an employee bar coded label, a patient bar coded label, a medicine bar coded label, and a medicine controller including a medicine controller bar coded label and a medicine administration device wherein the employee bar coded label, the patient bar coded label, the medicine bar coded label, and the medicine controller bar coded label are arranged differently; and
an administration monitoring system comprising a computer for medication, a barcode reader, and at least one handheld mobile device;
wherein the computer for medication is stored with a plurality of EMRs and electrically connected to the barcode reader and the handheld mobile device respectively;
wherein the administration monitoring system is connected to the medicine controller via the Internet; and
wherein the barcode reader reads barcodes on each of the employee bar coded label, the patient bar coded label, the medicine bar coded label, and the medicine controller bar coded label and sends the read barcodes to the computer for medication; a volume of substance to be administered is set by the medicine controller based on the read barcodes sent from the computer for medication via the handheld mobile device; the medicine controller outputs the set volume of substance to the computer for medication; the computer for medication compares the set volume of substance with a volume of substance written on the EMR, and a comparison result is sent to the handheld mobile device for showing.

2. The system for monitoring administered liquid substance of claim 1, wherein the volume of substance to be administered is set by the medicine controller based on substance, volume, usage, and times of administration written on the EMR.

3. The system for monitoring administered liquid substance of claim 1, wherein details of the EMR and printed barcodes read by the barcode reader are shown on the handheld mobile device.

4. The system for monitoring administered liquid substance of claim 1, wherein the handheld mobile device is a smart phone, a personal computer, a tablet computer, or a personal digital assistant (PDA) reader.

5. The system for monitoring administered liquid substance of claim 1, wherein the medicine controller is a flow controller device.

6. The system for monitoring administered liquid substance of claim 1, wherein the handheld mobile device shows IV infusion monitored by the administration monitoring system in real time and an alert of “infusion to be finished” is sent to the handheld mobile device for showing when the administration monitoring system monitors same; wherein the handheld mobile device shows system information; and wherein the handheld mobile device shows administration details, abnormal items, normal items, wrong medicine taking, medical employee changes, EMR changes, medicine volume changes, and serial numbers.

7. The system for monitoring administered liquid substance of claim 1, wherein the barcode reader is either a smart phone or a barcode scanner.

8. The system for monitoring administered liquid substance of claim 1, wherein the medicine controller is connected to the administration monitoring system by wire or wirelessly.

9. The system for monitoring administered liquid substance of claim 1, wherein the barcode reader, the handheld mobile device, and the computer for medication are connected together by wire or wirelessly.
